SEQUENCE LISTING (16 sequences)

SEQ ID NO: 1 - DNA, 12 bp, Artificial Sequence
  Random insert of CDR3 region from peptide library - Ab1 DNA
attagtagtgat

SEQ ID NO: 2 - protein, 4 aa, Artificial Sequence
  CDR3 random insert of Ab1
ISSD

SEQ ID NO: 3 - DNA, 33 bp, Artificial Sequence
  CDR3 random insert of Ab2 DNA
gcttttatgaagagtggtaagcgttttattcat

SEQ ID NO: 4 - protein, 11 aa, Artificial Sequence
  CDR3 random insert sequence of Ab2
AFMKSGKRFIH

SEQ ID NO: 5 - DNA, 33 bp, Artificial Sequence
  CDR3 random insert sequence of Ab3 DNA
cattttcattataagggtaagcttcagacgttt

SEQ ID NO: 6 - protein, 11 aa, Artificial Sequence
  CDR3 random insert of Ab3
HFHYKGKLQTF

SEQ ID NO: 7 - protein, 8 aa, Artificial Sequence
  SV40-NLS with extra C
PKKKRKVC

SEQ ID NO: 8 - protein, 9 aa, Artificial Sequence
  SV40-NLS-reverse with extra C
CVKRKKKPG

SEQ ID NO: 9 - protein, 20 aa, Artificial Sequence
  VprN with extra C
CNEWTLELLEELKNEAVRHF

SEQ ID NO: 10 - protein, 21 aa, Artificial Sequence
  VprC with extra C
CRHSRIGVTRQRRARNGASRS

SEQ ID NO: 11 - protein, 20 aa, Artificial Sequence
  VprN mutant with extra C
CNEATLELLPELKNPAVRHF

SEQ ID NO: 12 - protein, 15 aa, Artificial Sequence
  ARM with extra C
CGRKKRRQRRRAHQN

SEQ ID NO: 13 - protein, 6 aa, Artificial Sequence
  Tat short NLS
CGRKKR

SEQ ID NO: 14 - DNA, 17 bp, Artificial Sequence
  primer LMB3
caggaaacagctatgac

SEQ ID NO: 15 - DNA, 17 bp, Artificial Sequence
  PCR primer fdSEQ
gaattttctgtatgagg

SEQ ID NO: 16 - protein, 20 aa, bacteriophage fd
AEGDDPAKAAFDSLQASATE

(19) United States
(12) Patent Application Publication
(10) Pub. No.: US 2006/0240028 A1
(43) Pub. Date: Oct. 26, 2006

(54) Anti-nls substances and uses thereof in nuclear import inhibition

(76) Inventors: Abraham Loyter, Jerusalem (IL); Adolf Graessman, Berlin (DE); Ahuva Nissim, Ramat Gan (IL); Alexander Krichevsky, Jerusalem (IL); Nechama Zakai, Jerusalem (IL)

Correspondence Address: John P. White;Cooper & Dunham, 1185 Avenue of the Americas, New York, NY, 10036, US

(21) Appl. No.: 10/511,990

(22) PCT Filed: Apr. 21, 2003
(86) PCT No.: PCT/IL03/00328

§ 371 Date: Jan. 17, 2005

(30) Foreign Application Priority Data
Apr. 22, 2002 (IL) 149279

(51) Int. Cl. A61K 39/42; A61K 39/21; C07K 14/16; C07K 16/10

(52) U.S. Cl. 424/160.1; 424/208.1; 530/350; 530/388.3

(57) ABSTRACT

The present invention relates to a substance that specifically binds a nuclear localization signal (NLS)-containing molecule. Said substance may be a naturally occurring, synthetic or recombinant antibody, or it may be a protein, a peptide or a small molecule. Preferably, said NLS-containing molecule is a HIV-1 protein, more preferably Vpr or Tat. In addition, the present invention relates to a composition or a vaccine comprising the substance of the invention, as well as methods of inhibiting viral infection through the administration thereof.

### FIELD OF THE INVENTION

The present invention relates to nuclear import inhibition. More specifically, the present invention relates to substances that specifically bind to nuclear localization signal (NLS)-containing molecules, and thereby are able to block their nuclear import.

### BACKGROUND OF THE INVENTION

All publications mentioned throughout this application are fully incorporated herein by reference, including all references cited therein.

During the last few years, it has become clear that the Human Immunodeficiency Virus-1 (HIV-1) is able to infect terminally differentiated non-dividing cells, such as macrophages and quiescent T-lymphocytes. The ability to infect such cells implies that the HIV-1 genome is able to cross the nuclear envelope, likely through the Nuclear Pore Complexes (NPC) of host cells [Simm, L. G. et al. (1993) J Virol 67(7), 3969-77; Lewis, P. et al. (1992) EMBO J 11, 3053-3058]. Following cell penetration, the HIV-1 particles are uncoated and the viral genome is converted into the preintegration complex (PIC). The PIC is then imported into the nuclei of HIV-1 infected cells by one or more of its karyophilic proteins [Bukrinsky, M. et al. (1992) PNAS USA 89(14), 6580-4].

The ability of HIV-1 to infect non-dividing cells distinguishes it from other retroviruses, such as the MLV (murine leukemia virus), which only infect proliferating cells. Indeed, PICs derived from many oncoretroviruses, including MLV, are non-karyophilic and therefore enter the nucleus only during mitosis following the breakdown of the nuclear envelope [Lewis, P. F., and Emerman, M. (1994) J. Virol. 68, 510-516].

In the case of HIV-1, at least three viral proteins have been proposed to be involved in nuclear import of the PIC, thus displaying partially redundant nuclear localization activity. These are the MA, the Vpr and the IN [Bouyac-Bertoia, M. et al. (2001) Mol Cell 7(5), 1025-35; Jenkins, Y. et al. (1998). J Cell Biol 143, 875-885; Depienne, C. et al. (2000) Exp Cell Res 260, 387-395; Bukrinsky, M. I. et al. (1993a) Nature 365, 666-669]. However, the respective contribution of each of these karyophilic proteins to the nuclear import of viral PIC is not fully understood. It appears that the role of Vpr in mediating nuclear import of the HIV-1 PIC is indirect and requires the co-participation of MA. [Popov, S. et al. (1998) EMBO J 17, 909-917; Haffar, O. K. et al. (2000) J Mol Biol 299, 359-68]. Interestingly, it has been shown that Vpr is required for neither nuclear import of the PIC nor for virus replication, when using Vpr-deficient HIV-1 mutants in cells such as growth arrested T-cells [Heinzinger, N. K. et al. (1994) Proc. Natl. Acad. Sci. USA 91, 7311-7315; Gallay, P. et al. (1997) Proc. Natl. Acad. Sci. USA 94, 9825-9830; Bukrinsky, M. et al. (1993b) PNAS USA 90, 6125-6129.; Popov (1998) id ibid.]. However, it has been suggested that in such cells the role of the viral PIC in promoting nuclear import has been played by cellular proteins such as hsp70 [Agostini, I. et al. (2000) Exp Cell Res 259, 398-403]. Recently, it has been reported that the Vpr protein induces transient and localized herniations in the nuclear envelope, thus maybe providing a portal for PIC entry into the nucleus [de Noronha, C. M. et al. (2001) Science 294(5544): 1105-8].

It should be emphasized that the current view on the involvement of the HIV-1 karyophilic proteins in nuclear import of the PIC is based mainly on the use of viral particles deleted or mutated in one of these proteins [Haffar (2000) id ibid.; Koostra, N. A., and H. Schuitemaker (1999) Virology 253(2), 170-180]. However, due to the limitations inherent to these types of studies, the requirement of the MA and Vpr proteins for nuclear import of the PIC is still controversial. In addition, conflicting data have been reported on the function of the HIV-1 integrase in mediating nuclear import of the viral PIC [Bouyac-Bertoia (2001) id ibid.; Haffar (2000) id ibid.; Petit, C. et al. (2000) J Virol 74(15), 7119-26.]. Thus, the exact role of the Nuclear Localization Signal (NLS) found within the HIV-1 MA, Vpr and IN proteins, and the function of each of these sequences in virus infection continues to be a matter of strong debate.

Hence, the precise cellular pathway that mediates nuclear import of the Vpr protein still remains an open question. Nonetheless, somewhat is known about which of the Vpr domains are involved in this process. Vpr is a small protein composed of 96 amino acids with a molecular weight of approximately 11 kDa [Yuan, X. et al. (1990) AIDS Res and Human Retroviruses 6(11), 1256-71; Baldrich-Rubio, E. et al. (2001) J Gen Virol 82(Pt5), 1095-106]. Several studies have shown that nuclear accumulation of Vpr may be promoted by both N- and C-terminal domains [Jenkins (1998) id ibid.].

In a previous study, the inventors have used peptides derived from the Vpr protein in order to better characterize its NLS region. Peptides corresponding to the N-(residues 17 to 34) and C-terminal (residues 77 to 96) regions were synthesized and designated as VprN and VprC, respectively. VprN, but not VprC peptides, promoted entry of covalently attached labeled BSA molecules into nuclei of permeabilized cells [Karni, O. et al. (1998) FEBS Let. 429:421-425]. Consistent with various other observations, the inventors' results indicated that the Vpr protein harbors a non-conventional negatively charged NLS in its N terminus [Jenkins (1998) id ibid.; Karni et al. (1998) id ibid.]. Nuclear import of VprN-BSA conjugates was found to be energy and temperature dependent, and was inhibited by wheat germ agglutinin (WGA), demonstrating that the observed nuclear import is an active process that occurs via the NPC [Karni et al. (1998) id ibid]. The non-conventional nature of the Vpr protein suggested that trying to experimentally block its nuclear import could be a very challenging task.

Tat is another karyophilic protein from HIV-1. It is active in the cell nucleus [Cullen, B. R. (1998) Cell 93, 685-692; Pollard, V. W., and M. H. Malim (1998) Annu. Rev. Microbiol. 52, 491-532; Cullen, B. R. (1995) Aids 9, S19-32] and plays a pivotal role in viral replication as well as in the progression to overt acquired immune deficiency syndrome (AIDS) [Pollard, V. W. and M. H. Malim (1998) id ibid; Cullen, B. R. (1995) id ibid; Cullen, B. R. (1993) Cell 73(3), 417-20; Choudhury, I., J. Wang, et al. (1998) Journal of acquired immunodeficiency syndromes and human retrovirology 17, 104-111].

Antibodies, proteins and peptides that specifically mask the NLS of HIV karyophilic proteins may offer an alternative approach for elucidating the role, as well as the relative contribution of these sequences to the nuclear import process of the HIV-1 PIC. Furthermore, such studies may help in gaining better understanding of the spatial rearrangement of the karyophilic proteins within the PIC or PIC-importins complexes and their relative susceptibility to external ligands. This approach may also be useful in obtaining anti-viral drugs.

Thus it is an object of the present invention to provide substances that can recognize and block Vpr and Tat nuclear import. Compositions comprising said substances and uses thereof are further objects of the present invention. The present invention also provides a method for inhibiting viral infection.

These and other objects of the invention will become more apparent as the description proceeds.

### SUMMARY OF THE INVENTION

The present invention relates to substances that specifically bind to nuclear localization signal (NLS)-containing molecules, and thereby are able to specifically block their nuclear import.

More specifically, the present invention relates to single chain fragments (scFv), proteins and peptides that specifically recognize the NLS of the HIV-1 Vpr and Tat proteins, and are able to block the nuclear import of Vpr and/or Tat. Thus, the invention also relates to uses of such fragments and peptides in blocking HIV infection, as well as to vaccines and compositions comprising the substances of the invention.

Thus, in a first aspect, the present invention relates to a substance that specifically binds a NLS-containing molecule, or functional fragments or derivatives thereof.

In one specific embodiment, said NLS-containing molecule is the HIV-1 protein Vpr. Alternatively, said NLS-containing molecule is the N-terminal domain of Vpr (amino acids 17-34). In either one, said substance has a CDR3 region containing an amino acid sequence of any one of SEQ. ID. NO. 1, SEQ. ID. NO.3, and SEQ. ID. NO.5, wherein said amino acid sequence is encoded by the nucleic acid sequence of SEQ. ID. NO. 2, SEQ. ID. NO.4, and SEQ. ID. NO. 6, respectively.

In another embodiment, said substance, or functional fragments or derivatives thereof, is selected from any one of a naturally occurring, synthetic or recombinant antibody, scFv, Fv, Fab′, Fab, diabody, linear antibody or F(ab′)₂ antigen binding fragment of an antibody, a peptide and a small molecule. Preferably said substance is a scFv, more preferably a recombinant scFv.

In another specific embodiment said NLS-containing molecule is the HIV-1 protein Tat and said NLS-binding substance is a bacteriophage fd protein p8-derived peptide.

In another aspect, the invention relates to a composition comprising at least one substance of the invention, and optionally further comprising pharmaceutically acceptable diluents, additives and carriers.

In a further aspect the present invention relates to a vaccine, wherein said vaccine comprises at least one substance of the invention, and optionally further comprises pharmaceutically acceptable diluents, additives and carriers.

In a yet further aspect, the present invention provides a method of inhibiting the import of a NLS-containing molecule into a nucleus of a cell, by contacting said cell with a substance as defined in the invention.

The present invention also provides a method of specifically inhibiting the import of the viral proteins Vpr and Tat, and a method of inhibiting the import of the pre-integration complex (PIC), into the nucleus of a cell. Both methods are effected by contacting said cell with at least one substance as defined in the invention.

In addition, the invention provides a method of inhibiting viral infection by administering a substance of the invention to an organism in need. Preferably, said organism is any one of a plant and a mammal.

In an additional aspect, the present invention provides a method of inhibiting cell proliferation, oncogenesis and autoimmune responses, by administering at least one of the substances defined by the invention to an organism in need. Preferably, said organism is a mammal.

Further, the invention also provides a method of conferring immunity against a viral infection, by administering to a subject in need a vaccine comprising a substance as defined by the invention.

Lastly, the invention relates to the use of a substance of the invention in the preparation of a pharmaceutical composition and in the preparation of a vaccine.

### BRIEF DESCRIPTION OF THE FIGURES

FIG. 1A-B: Binding of selected phases to BSA-VprN and other various BSA-NLS conjugates

FIG. 1A: Binding was estimated by the ELISA method. Column numbers indicate individual phage preparations. Rows on the left indicate BSA-NLS conjugates. The OD values were determined by an ELISA reader and converted to a gray scale. Note that dark and white fields indicate strong and no binding, respectively; gray fields indicate binding of intermediate strength. Phages 1-4 and 11 exhibited strong and specific binding to the VprN sequence.

FIG. 1B: Specific binding to BSA-VprN of the three selected phages used (black, phage A; gray, phage B; white, phage C). All three phages specifically bind the BSA-VprN conjugate, but do not react with the BSA-VprN “mutant” or the BSA-SV40 NLS.

Abbreviations: pep. conj., peptide conjugates; mut., mutant; seq., sequence; sh., short; ag., antigen; lg., large; rev., reverse; ph., phage; col., colonies.

FIG. 2A-B: Interaction of the anti-VprN peptides with the BSA-NLS conjugate.

FIG. 2A Estimation of the binding ability of the anti-VprN peptides to various BSA-NLS conjugates (black, peptide a; white, peptide b; gray, peptide c).

FIG. 2B: Dependency of the binding to BSA-VprN conjugate on the anti-VprN peptide concentration (diamond, peptide a; square, peptide b; triangle, peptide c). Binding was estimated by ELISA assay.

Abbreviations: pept., peptide; mut., mutant; lg., large; ag., antigen.

FIG. 3: SDS-PAGE of the purified anti-VprN scFv antibodies.

Ab1, Ab2 and Ab3 were analyzed using a 12% polyacrylamide gel. Ab1 is of a lower molecular weight due to the fact that its random CDR3 insert consists of only 4 amino acids, as opposed to 10 amino acids in the CDR3 of Ab2 and Ab3.

FIG. 4A-B: Binding of the anti-VprN scFv Antibodies to BSA-VprN conjugate.

FIG. 4A: Binding of Ab1, Ab2 and Ab3 to various BSA-NLS conjugates (black, peptide a; white, peptide b; gray, peptide c).

FIG. 4B: Dependency of binding to BSA-VprN on antibody concentration (diamond, peptide a; square, peptide b; triangle, peptide c).

Abbreviations: antib., antibody; ag., antigen; mut., mutant; lg., large.

FIG. 5A-C: Specific inhibition of the binding of the anti-VprN scFv antibodies to a surface bound BSA-VprN conjugate by soluble BSA-VprN conjugates.

FIG. 5A: Ab1.

FIG. 5B: Ab2.

FIG. 5C: Ab3.

Abbreviations: mut., mutant; conc., concentration.

FIG. 6A-C: Specific inhibition of the interaction between the anti-VprN scFv antibodies and a surface-bound BSA-VprN conjugate.

Diamond, VprN peptide; triangle, SV40 large Tag NLS peptide; square, VprN mutant peptide.

FIG. 6A: Ab1.

FIG. 6B: Ab2.

FIG. 6C: Ab3.

Abbreviations: mut., mutant; pept. conc., peptide concentration.

FIG. 7A-D: Inhibition of VprN-mediated nuclear import by anti-VprN scFv Ab2 in permeabilized HeLa cells.

FIG. 7A: Nuclear import was followed by fluorescence microscopy using FL-BSA-VprN as transport substrate.

FIG. 7B: As in (A), using Ab2 at a ratio of Ab:transport substrate of 2:1.

FIG. 7C: As in (A), using FL-BSA-SV40 NLS as transport substrate.

FIG. 7D: As in (C), using Ab2 at a ratio of Ab:transport substrate of 2:1. Nuclear transport is observed in A, C and D, but not in B.

FIG. 8: Quantitative estimation of inhibition of Vpr-mediated nuclear import by anti-VprN scFv Ab2.

Diamond, biotinylated BSA-SV40 NLS conjugate; square, biotinylated BSA-VprN conjugate.

Abbreviations: syst., system; Perc. Nucl. Ent., percentage of nuclear entry.

FIG. 9A-D: Inhibition of VprN-mediated nuclear import by anti-VprN scFv Ab2 in microinjected COS cells.

FIG. 9A: FL-BSA-SV40 NLS.

FIG. 9B: Mixture of FL-BSA-VprN with Ab2 at a 1:2 ratio, respectively.

FIG. 9C: FL-BSA-SV40 NLS.

FIG. 9D: FL-BSA-SV40 NLS with Ab2 at the same ration as in (B).

Nuclear import can be observed in A, C and D, but not in B.

FIG. 10: Binding of the anti-VprN scFv antibodies to the Vpr-GST fusion protein.

Binding of the antibodies to surface bound VprN-GST was estimated by ELISA. Black, Ab1; white, Ab2; gray, Ab3.

FIG. 11A-B: Binding of the anti-VprN scFv antibodies to the full-length Vpr-GST protein and specific inhibition by VprN peptides and their BSA conjugates.

FIG. 11A: BSA-VprN (continuous line) or BSA-VprN mutant (dashed line) conjugates were added to the antibodies solution. After incubation with the surface-bound Vpr-GST protein, the amount of antibodies bound to Vpr-GST was estimated by ELISA.

FIG. 11B: VprN (continuous line) or VprN mutant (dashed line) peptides were added to the antibodies solution. After incubation with the surface-bound Vpr-GST protein, the amount of antibodies bound to Vpr-GST was estimated by ELISA.

Square, Ab1; diamond, Ab2; triangle, Ab3.

Abbreviations: conc., concentration; mut., mutant.

FIG. 12 A-D: Inhibition of FL-Vpr-GST nuclear import by a scFv anti-VprN antibody in digitonin permeabilized HeLa cells.

FIG. 12A: FL-Vpr-GST.

FIG. 12B: FL-Vpr-GST mixed with Ab2 at a 1:4 ratio.

FIG. 12C: FL-Vpr-GST with WGA

FIG. 12D: FL-Vpr-GST in the presence of 5× excess unlabeled Vpr-GST.

FIG. 13A-C: Specificity of Tat NLS binding to fd bacteriophage and NTP8.

FIG. 13A: Graph showing the binding of fd phage to various surface immobilized proteins and BSA-NLS conjugates.

FIG. 13B: Graph showing the NTP8-biotin peptide binding to various surface immobilized proteins.

FIG. 13C: Graph showing the inhibition of NTP8-biotin binding to surface bound BSA-Tat NLS by fd particles.

Abbreviations: surf. bd. prot., surface bound protein.

FIG. 14A-F: Nuclear import of FL-BSA-Tat conjugates in HeLa cells

FIG. 14A: Nuclear import of FL-BSA-Tat.

FIG. 14B: Nuclear import of FL-BSA-Tat at 4° C.

FIG. 14C: As in A, with WGA added to final concentration of 1 mg/ml.

FIG. 14D: As in A, with 10⁹-10⁸ of phage cfu added.

FIG. 14E: As in A, with SV40 T-antigen NLS peptide added (ratio of 20/1 of SV40 T-antigen NLS peptide/FL-BSA-Tat).

FIG. 14F: As in A, with Tat-NLS peptide added (ratio of 20/1 of Tat-NLS peptide/FL-BSA-Tat).

FIG. 15A-D Nuclear import of FL-BSA-VprN conjugate in permeabilized HeLa cells.

FIG. 15A: Nuclear import of FL-BSA-VprN.

FIG. 15B: Nuclear import of FL-BSA-VprN at 4° C.

FIG. 15C: As in A, with WGA added to final concentration of 1 mg/ml.

FIG. 15D: As in A, with 10⁹-10⁸ of phage cfu added.

### DETAILED DESCRIPTION OF THE INVENTION

The following abbreviations have been used throughout this patent application:
Ab: antibody
ARM: Arginine rich motif
GST: glutathione S-transferase
NLS: nuclear localization signal
NPC: nuclear pore complex
PIC: pre-integration complex
scFv: single-chain variable fragment
Vpr: viral protein r
WGA: wheat-germ agglutinin

The present invention relates to substances that specifically bind to nuclear localization signal (NLS)-containing molecules, and thereby are able to specifically block nuclear import of the same.

NLS is a specific domain present in a variety of proteins [see e.g. Goldfarb, D., and N. Michaud (1991) Trends Cell Biol. 1, 20-24; Gorlich, D., and I. W. Mattaj (1996) Science 271, 1513-1518] characterized by its capacity to direct the protein to the nucleus of the cell. Interestingly, most NLSs do not consist of a consensus sequence, although the NLS of SV40 large T antigen provides the prototypic monopartite NLS [Schneider, J. et al. (1988) Cell 54, 117-125]. Thus, NLSs are specific to the protein to which they are part of. Consequently, blocking the NLS of one protein should not interfere with the nuclear import of other proteins, and is therefore specific to the protein in question.

In the present invention the inventors have used a phage display ScFv antibody library for obtaining anti-NLS ScFv fragments (antibodies) and peptides, specifically anti-Vpr ScFv antibodies and peptides. For that purpose, the N-terminus domain of the HIV-1 Vpr protein was used as the target for the phage display library. The isolated scFv antibodies exhibited high and specific binding to the VprN peptide, and were able to inhibit nuclear import mediated by this peptide. Furthermore, the anti-VprN scFv fragments (antibodies) also recognized the full length recombinant Vpr-GST protein, and inhibited its nuclear import as well. In addition, the inventors have shown that the p8 protein of the fd bacteriophage can bind the NLS of the HIV-1 Tat protein.

Thus, in a first aspect, the present invention relates to a substance that specifically binds a NLS-containing molecule, or functional fragments or derivatives of said molecule. Said substance may be cell-permeable. Alternatively, said NLS-binding molecule may be originally a non-permeable substance which is converted into permeable by suitable means.

In one embodiment, said substance, or functional fragments or derivatives thereof is selected from any one of a naturally occurring, synthetic or recombinant antibody, scFv, Fv, Fab′, Fab, diabody, linear antibody or F(ab′)₂ antigen binding fragment of an antibody, a protein, a peptide and a small molecule. Preferably said substance is a scFv, more preferably a recombinant scFv.

Alternatively, said substance is a peptide derived from the p8 protein of bacteriophage fd. Preferably, said peptide comprises the first 20 amino acids of p8, and has the amino acid sequence SEQ. ID NO.16.

It is to be understood that by fragment, functional fragment or derivative of a substance of the invention as used herein is meant any such fragment or derivative that is able to bind to at least a NLS of a NLS-containing molecule. In addition, derivatives of a substance of the invention may also be a dimer, trimer or polymer of the substance of the invention.

More specifically, the present invention relates to single chain fragments (scFv), proteins and peptides that specifically recognize the NLS of the HIV-1 Vpr protein and/or HIV-1 Tat protein, and are able to block the nuclear import of Vpr and/or Tat. Thus, the invention also relates to uses thereof in blocking HIV infection, as well as vaccines and compositions comprising the invention.

The choice of the VprN peptide as a target for screening with the scFv library was based on the hypothesis that it should harbor an NLS, since it was able to promote nuclear import of BSA into the nuclei of permeabilized cells [Jenkins (1998) id ibid.; Karni et al. (1998) id ibid.]. The VprN synthetic peptide, comprising amino acids 17-34 from the N-terminal domain of the HIV-1 Vpr protein [www.expasy.ch, SWISS-PROT and TrEMBL—Protein knowledgebase, Accession No. Q9EAL0], has been suggested to possess an α-helical structure probably similar to its structure within the intact protein [Luo, Z. et al. (1998) BBRC 244, 732-736; Wecker, K, and B. P. Roques (1999) Eur J Biochem 266, 359-369].

Therefore, the VprN peptide is a convenient target for obtaining complementary antibodies or peptides from phage display libraries, since it bears a relatively stable α-helical ternary structure. It is noteworthy that the phage particles within the library used here possess one or none antibody fragment per particle thus allowing selection of antibodies with relatively high affinities.

Hence, in one specific embodiment, said NLS-containing molecule is the HIV-1 protein Vpr. Alternatively, said NLS-containing molecule is the N-terminal domain of Vpr (amino acids 17-34). In either one, said substance has a CDR3 region containing an amino acid sequence of any one of SEQ. ID. NO. 1, SEQ. ID. NO.3, and SEQ. ID. NO.5, wherein said amino acid sequence is encoded by the nucleic acid sequence of SEQ. ID. NO. 2, SEQ. ID. NO.4, and SEQ. ID. NO. 6, respectively.

Interestingly, no homology between the sequences corresponding to the random CDR3 inserts of the three anti-VprN scFv fragments (antibodies) of the invention could be determined. Moreover, synthetic peptides whose amino acid composition was based on the sequence of the random CDR3 inserts of three arbitrary chosen VprN binders either failed to bind the VprN peptide or showed low binding activity (Example 2). Thus, it is possible that in addition to CDR3, other CDR loops may participate in the antibody-mediated binding of the phage particles to VprN. Therefore, it is likely that inhibitors of the VprN-mediated nuclear import of smaller molecular weight might increase the binding abilities of the anti-VprN peptides.

In contrast to the properties of the anti-VprN peptides, the three specific anti-VprN scFv fragments (antibodies) of the invention expressed by anti-VprN phage particles possessed high and specific binding activity to the VprN peptide. The anti-VprN ScFv fragments were specific since they were able to interact with the VprN peptide, but not with a peptide bearing the VprN “mutant” sequence, which differs from its parental peptide by only three amino acids. Furthermore, the anti-VprN scFv antibodies failed to recognize other NLS's, such as the SV40 NLS or the Tat ARM (Example 3). Interestingly, the fragments (antibodies) of the invention interacted also with full length recombinant Vpr-GST protein, probably recognizing its VprN domain (residues 17-34). Evidently, this should be inferred from the results demonstrating that the interaction between the fragments (antibodies) and the recombinant Vpr-GST protein can be inhibited by the VprN peptide and its BSA conjugate (Example 5).

The scFv fragments (antibodies) of the invention, encoded by SEQ. ID. NO.2, SEQ. ID. NO.4 and SEQ. ID. NO.6, are designated herein as Ab1, Ab2 and Ab3, respectively.

In a second specific embodiment, said NLS-containing molecule is the HIV-1 protein Tat.

As an essential regulatory protein of HIV-1, Tat function involves direct interaction with an RNA target, termed TAR, that is mediated by an arginine-rich RNA binding motif (ARM) in Tat that also functions as a Nuclear Localization Signal (NLS) [Truant, R., and B. R. Cullen (1999) Mol Cell Biol 19, 1210-1217].

Hence specifically, when said NLS-containing molecule corresponds to Tat, or to the ARM sequence of Tat, said substance that specifically binds said NLS is the p8 protein of bacteriophage fd.

As shown in Examples 7 and 8, a peptide containing the first twenty amino acids of p8, which in these specific examples was synthesized by the inventors and designated NTP8 (SEQ. ID. NO.16), was able to interact with immobilized Tat-BSA conjugates and inhibit its entry into the cell nucleus.

Thus, fd bacteriophage p8 can inhibit Tat-ARM mediated nuclear import.

In another aspect, the invention relates to a composition comprising at least one substance of the invention, and optionally further comprising pharmaceutically acceptable diluents, additives and carriers.

The preparation of pharmaceutical compositions is well known in the art and has been described in many articles and textbooks, see e.g., Gennaro A. R. ed. (1990) Remington's Pharmaceutical Sciences, Mack Publishing Company, Easton, Pa., and especially pages 1521-1712 therein.

In a further aspect the present invention relates to a vaccine, wherein said vaccine comprises at least one substance of the invention or a mixture thereof, and optionally further comprising pharmaceutically acceptable diluents, additives and/or carriers.

By pharmaceutically acceptable (or physiologically acceptable) additive, carrier and/or diluent is meant any additive, carrier or diluent that is non-therapeutic and non-toxic to recipients at the dosages and concentrations employed, and that does not affect the pharmacological or physiological activity of the active agent.

In a yet further aspect, the present invention provides a method of inhibiting the import of a NLS-containing molecule into a nucleus of a cell, by contacting said cell with a substance as defined in the invention.

In particular, the present invention provides a method of specifically inhibiting the import of the viral proteins Vpr and/or Tat, and a method of inhibiting the import of the pre-integration complex (PIC), into the nucleus of a cell. Both methods are effected by contacting cells with at least one substance of the invention.

The ability of the anti-VprN scFv fragments (antibodies) to inhibit nuclear import mediated by either by the VprN peptide or the recombinant Vpr-GST protein was highly dependent on the antibody/VprN ratio. At high ratios, almost total inhibition of nuclear import was observed. The inhibition of nuclear import, as well as the antibodies' binding to VprN was highly specific, since inhibition was observed only with VprN but not with the SV40 NLS. Surprisingly, the anti-VprN antibodies blocked import not only into nuclei of permeabilized HeLa or Colo-205 cells, but also in microinjected, non-permeabilized COS cells. The results shown in Example 4 strongly indicate that the inhibition observed is due to specific masking of the VprN moiety by the antibody and thus preventing its interaction with a putative cellular receptor. Curiously, the inhibition observed in the microinjected cells suggests that the intracellular environment did not promote dissociation of the antibody-VprN complex, thus indicating high binding affinity between these two moieties.

The fact that all three anti-VprN ScFv fragments (antibodies) blocked nuclear import of the full-length protein was unexpected. Although both Ab2 and Ab3 interacted with the full length Vpr protein to the same extent as with the VprN-BSA conjugate (Example 5), Ab1 showed lower binding affinity to the Vpr protein when compared to that obtained with the VprN-BSA conjugate. This may indicate that the epitope within the VprN domain recognized by Ab1 differs from that recognized by the two other antibodies. Together with the results of Example 2, wherein the synthetic peptides derived from the CDR3 insert of the anti-VprN scFv antibodies were unable to inhibit VprN nuclear import, it seemed unlikely that Ab1, Ab2 and Ab3 would be able to inhibit nuclear import, moreover so efficiently.

NLSs are also known to be involved in the nuclear transport of nucleic acids. After viral infection (i.e., entering into the cell), the genetic material of viruses has to make its way to the nucleus, where it will integrate into the host's DNA and be transcribed. NLSs have been shown to bind and transport RNA [Goldfarb (1991) id ibid.]. This latter function is particularly relevant during virus infection and assembly of viral particles, in the case of for example influenza virus, herpes virus, plant viruses (such as the Gemini virus), adenovirus, amongst others.

So in addition, the invention provides a method of inhibiting viral infection by administering a substance of the invention to an organism in need. Preferably, said organism is any one of a plant and a mammal.

Yet another function of NLSs is their involvement in the nuclear transport of proteins other than viral. For example, the NLS motif is also found in transcription factors [Baeuerle, P. A., and D. Baltimore (1988) Science 242, 540-546], which are, as all proteins, synthesized in the cytoplasm, but need to perform their function in the nucleus, and thus their necessity to undergo nuclear import. Many transcription factors have been shown to be involved in cell proliferation and differentiation, and consequently their de-regulation can lead to ongenicity. Thus, inhibition of the nuclear import of such transcription factors could result in inhibition of malignancy.

Inhibition of NLS function could also be relevant for the inhibition of auto-immune processes, since NFκB, a transcription factor shown to be involved in auto-immunity [Baeuerle (1988) id ibid.], requires its NLS for its function.

In view of these additional functions of the NLS, it is an additional aspect of the present invention to provide a method of inhibiting cell proliferation, oncogenesis and autoimmune responses in a cell, by administering at least one of the substances defined by the invention to an organism in need. Preferably, said organism is a mammal.

Further, the invention also provides a method of conferring immunity against a viral infection, by administering to a subject in need a vaccine comprising a substance as defined by the invention.

When a vaccine composition of the present invention is used for administration to an individual, it can further comprise salts, buffers, adjuvants, or other substances which are desirable for improving the efficacy of the composition. Adjuvants are substances that can be used to augment a specific immune response. Normally, the adjuvant and the composition are mixed prior to presentation to the immune system, or presented separately, but into the same site of the mammal being immunized. Examples of materials suitable for use in vaccine compositions are provided in Genaro et al. eds. (1990) id ibid.

In an additional aspect, the invention provides a method for in vitro screening for infectivity of viral particles and/or nuclear import of proteins, wherein said method comprises the steps of:
(a) contacting cells with a substance of the invention;
(b) detecting the intracellular localization of said virus, viral particle and/or protein.

Lastly, the invention relates to the use of a substance of the invention in the preparation of pharmaceutical compositions and in the preparation of vaccines.

The pharmaceutical compositions and vaccines of the present invention, suitable for inoculation or for parenteral or oral administration, comprise at least one substance of the invention, and optionally further comprise sterile aqueous or non-aqueous solutions, suspensions, and emulsions. The composition can further comprise auxiliary agents or excipients, as known in the art. See, e.g., Berkow et al., eds. (1987) The Merck Manual, 15^{th} edition, Merck and Co., Rahway, USA; Goodman et al., eds. (1990) Goodman and Gilman's The Pharmacological Basis of Therapeutics 8^{th} ed., Pergamon Press, Inc., Elmsford, USA; Williams and Wilkins (1987) Avery's Drug Treatment: Principles and Practice of Clinical Pharmacology and Therapeutics, 3^{rd} ed., ADIS Press, LTD., Baltimore, USA; Genaro et al. eds. (1990) Remington's Pharmaceutical Sciences, Mack Publishing Co, Easton, USA, pp. 1324-1341; Katzung, ed. (1992) Basic and Clinical Pharmacology, 5^{th} ed., Appleton and Lange, Norwalk, USA.

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents. See, e.g., Berkow (1987) id ibid.; Goodman (1990) id ibid.; Williams and Wilkins (1987) id ibid.; Genaro (1990) id ibid.; and Katzung (1992) id ibid., which are entirely incorporated herein by reference, included all references cited therein.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Experimental Procedures

DNA sequence of the corresponding random insert within the CDR3 of Ab1, Ab2 and Ab3:

| | |
|---|---|
| Ab1: | |
| ATTAGTAGTGAT | (SEQ. ID. NO.1) |
| Ab2: | |
| GCTTTTATGAAGAGTGGTAAGCGTTTTATTCAT | (SEQ. ID. NO.2) |
| Ab3: | |
| CATTTTCATTATAAGGGTAAGCTTCAGACGTTT | (SEQ. ID. NO.3) |

Amino acid sequence of the corresponding CDR3 of Ab1, Ab2 and Ab3

| | |
|---|---|
| Ab1: ISSD | (SEQ. ID. NO.4) |
| Ab2: AFMKSGKRFIH | (SEQ. ID. NO.5) |
| Ab3: HFHYKGKLQTF | (SEQ. ID. NO.6) |

Synthesis of Peptides

The following peptides were synthesized as described before [Karni et al. (1998) id ibid.]:
a) ¹²⁶PKKKRKV¹³²C (SEQ. ID. NO.7), the SV40 large T-antigen NLS (SV40-NLS);
b) C¹³²VKRKKKPG¹²⁶ (SEQ. ID. NO.8), a peptide bearing the SV40 large T-antigen NLS in a reverse order (‘reverse’);
c) C¹⁶NEWTLELLEELKNEAVRHF³⁴ (SEQ. ID. NO.9), a peptide derived from the N-terminus of the Vpr protein (VprN);
d) C⁷⁷RHSRIGVTRQRRARNGASRS⁹⁶ (SEQ. ID. NO.10), a peptide derived from the C-terminus of the Vpr protein (VprC);
e) C¹⁶NEATLELLPELKNPAVRHF³⁴ (SEQ. ID. NO.11), a VprN mutant;
f) C⁴⁸GRKKRRQRRRAHQN⁶¹ (SEQ. ID. NO.12), the HIV-1 Tat-NLS (the ARM sequence);
g) C⁴⁸GRKKR⁵² (SEQ. ID. NO.13), Tat “short” NLS.

Cysteine residues were added to the N- or C-terminus of the original NLS sequences. Peptides derived from the CDR3 random insert region of anti-VprN scFv antibodies (SEQ. ID. NO.4, SEQ. ID. NO.5 and SEQ. ID. NO.6) were synthesized using the same procedure. Where needed the peptides were labeled with biotin at the N- or C-terminus.

Chemical Conjugation of the Synthetic Peptides to BSA, Fluorescence-Labeled BSA (FL-BSA) and Biotinilated BSA

Chemical conjugation of the synthetic peptides to BSA molecules (Sigma, No. A-7906) was conducted as described before [Adam, S. A. et al. (1992) Methods in Enzymology 219, 97-110; Broder, Y. C. et al. (1997) FEBS Lett. 412, 535-539]. Briefly, the various peptides were conjugated to rhodamine (lissamine rhodamine B sulfonyl chloride, mixed isomers, from Molecular Probes). Labeling was performed according to the manufacturer instructions. Biotin-labeled BSA molecules were purchased from Sigma. The conjugates obtained were concentrated using a VivaScience concentrator (Sartorius), MWCO 50,000.

Panning and Screening of the Phage Display scFv Library

This protocol is schematically represented as follows:

| | |
|---|---|
| Step I: | ScFv antibody library |
| | panning on BSA-VprN |
| | conjugates |
| | ↓ |
| Step II: | Panning of the eluate |
| | from step I on BSA molecules |
| | ↓ |
| Step III: | Screening of the phages |
| | from the supernatant of |
| | step II on BSA-VprN |
| | conjugates (ELISA) |
| | ↓ |
| Step IV: | Screening of positive |
| | clones from step III |
| | on a variety of BSA-NLS |
| | conjugates |
| | ↓ |
| Step V: | Determination of amino |
| | acid sequences of the |
| | scFv domains in specific |
| | BSA-VprN binders |
| | ↓ |
| Step VI: | Purification of the scFv Abs |
| | and synthesis of peptides |
| | bearing the CDR3 random |
| | insert sequences from the |
| | three arbitrary chosen |
| | BSA-VprN binding phages |

Panning of the human scFv antibody phage display library [Harrison, J. L. et al. (1996) Methods in Enzymology 267, 83-109; Nissim, A. et al. (1994) EMBO J 13, 692-698] was conducted as follows. Two immunotubes (5 ml, NUNC) were incubated in parallel, with rotation at room temperature overnight, one containing the BSA-VprN conjugate and the other one containing only BSA, both in 0.1 mg/ml carbonate buffer (0.05M Na₂CO₃/0.05M NaHCO₃, pH9.6). Following incubation, the tube with the surface attached BSA-VprN conjugates was washed three times with 1× PBS and then incubated for two hours at 37° C. with 2% (w/v) skim milk (DIFCO) in 1×PBS (2% MPBS). After three washes with 1× PBS, the scFv phage library (4 ml in 1% MPBS) was added and the tube containing the phage particles was incubated for 30 min with, and for 1.5 hours without rotation, at room temperature. The supernatant was then removed and the tube was washed ten times first with 1×PBS containing 0.05% Tween 20 and then with 1×PBS only. For elution of bound phages, 1 ml of 0.1M triethylamine was added, incubated for 10 min at room temperature with rotation, and the triethylamine solution containing the eluted phages collected and mixed with 0.5 ml of 1M Tris-HCl, pH7.4. In parallel, the second tube, containing surface-attached BSA molecules was washed with 1×PBS, incubated with 2% MPBS, and again washed with 1×PBS as described above for the BSA-VprN containing tube. The solution containing the eluted phages was then added to this tube and incubated for 30 min with, and for 1.5 hours without, rotation at room temperature. The supernatant was collected and used for further screenings using an ELISA assay system as described [Harrison (1996) id ibid.].

PCR for Determination of CDR3 Random Insert Sequences and VH Typing of the scFv Antibodies

The CDR3 random insert sequence and the VH type of the scFv antibodies were elucidated through PCR analysis of the scFv domain. DNA from phage infected bacteria was subjected to several rounds of amplification with 100 pM of LMB3 (SEQ. ID. NO.14, 5′CAGGAAACAGCTATGAC3), and fdSEQ (SEQ. ID. NO.15, 5′GAATTTTCTGTATGAGG3) primers. The PCR products were purified using High Pure PCR Product Purification Kit (purchased from Boehringer Manheim, Germany), according to manufacturers instructions. Purity and size (around 900 bp) of the products were verified by agarose gel electrophoresis.

Expression and Purification of Soluble anti-VprN scFv Antibodies

Expression of soluble scFv in phage infected HB2151 bacteria was performed as described before [Harrison (1996) id ibid.]. The expressed anti-VprN scFv antibodies were purified using CNBr-activated Sepharose 4 Fast Flow (from Pharmacia) column with covalently linked VprN-BSA conjugates. Covalent attachment of the VprN-BSA conjugates to the resin was performed according to manufacturer instructions. The expressed antibody preparations were loaded on the column and then incubated with rotation for 1 hour at 4° C. The column was washed successively first with PBS secondly with PBS-0.5M NaCl, thirdly with 0.2M Glycine, pH6.0 and finally with 0.2M Glycine, pH5.0; each time with 5× column volumes. Elution of the antibody was performed in batch by incubating with 3× column volumes of 0.2M Glycine, pH3.0 for 20-30 min at 4° C. with rotation. The eluant obtained was immediately mixed with 1M Tris-HCl, pH7.4, dialyzed against 0.1×PBS and then concentrated 10× in the SpeedVac Concentrator. SDS-PAGE of the ScFv antibodies was performed as described [Laemmli, U.K. (1970) Nature 277, 680-685], with Mini-PROTEAN 3 Cell (from BIO-RAD), using 12% stacking and resolving gels and 0.75 μg of protein per sample.

ELISA Assays

The various ELISA assays used in the present work were carried out as follows: MaxiSorb plates (NUNC) were covered overnight with 25-100 μg/ml of the antigen (in Carbonate Buffer 0.05M Na₂CO₃/0.05M NaHCOs, pH9.6) at 4° C. On the next day, the plates were washed three times with 1×PBS and blocked with 2% BSA in 1×PBS (for antibodies) or 2% Skim Milk (DIFCO) in 1×PBS (for all other molecules) for 2 hours at 37° C. The plates were then washed three times with 1×PBS, and the appropriate ligand (in blocking solution) was added and incubated for 2 hours at 37° C. The plates were then washed three times with 1×PBS and the following detecting agents were added for detecting the antigen-ligand complexes: Avidin-POD (Roche Diagnostics), for biotin labeled molecules (1 unit/ml); anti-M13 monoclonal antibody (Amersham Pharmacia, 1:5000), for the bound phages; and a mix of mouse anti-myc antibody (Sigma, 1:1000) with anti-mouse-HRP antibody (Sigma, 1:2000) for the scFv antibodies. Each detecting agent was dissolved in the appropriate blocking solution (see above). Following incubation for 1 hour at 37° C. with the detecting agent, the plates were washed three times with 1×PBS and the appropriate substrate was added, according to manufacturer instructions.

Cell Culture

Colo-205 (human colon adenocarcinoma cells, ATCC CCL 222) and HeLa cells were maintained in RPMI 1640 and DMEM media respectively, supplemented with 10% FCS, 0.3 g/l L-glutamine, 100 U/ml penicillin and 100 U/ml streptomycin (Beit Haemek, Israel), as previously described [Broder (1997) id ibid.].

Estimation of Nuclear Import by Fluorescence Microscopy

Nuclear import of rhodamine-labeled BSA-NLS conjugates (FL-BSA-NLS) was performed as described before [Adam (1992) id ibid.; Broder (1997) id ibid.]. Amounts of FL-BSA-NLS substrate for FL-BSA SV40 large T-Ag NLS was 3-5 μg/system and for the FL-BSA-VprN 25 μg/system. Controls included: 1 mg/ml Wheat Germ Agglutinin (WGA), 4 mM of GTP-γ-S, or 1000 units/ml of Hexokinase and 10 mM Glucose. Samples were observed and photographed using confocal fluorescent microscopy.

Quantitative Estimation of Nuclear Import

Nuclear import was quantitatively determined by the ELISA based method as described before [Friedler, A. et al. (1998) Biochemistry 37, 5616-5622] with the following modifications. Colo-205 cells were permeabilized using 1 ml of 40 μg/ml digitonin (Fluka) per 10⁷ cells as follows: half of the volume of the digitonin solution was applied and the rest was added gradually in portions of 100-200 μl. After each addition a sample of cells was examined by phase microscopy. When 70-80% of the cells appeared permeabilized, as observed by light microscopy, the process was stopped by 50-100× dilution with cold transport buffer.

Microinjection of BSA-NLS Conjugates into Cultured COS Cells

Microinjection into cultured cells was performed exactly as described [Graessmann, M. and A. Graessmann (1983) Methods Enzymol. 101, 482-92).

Expression and Purification of Vpr-GST Fusion Protein

E. coli bacteria (strain AN3347) carrying a plasmid bearing the Vpr-GST fusion protein [Piller, S. C. et al. (1996) Proc Natl Acad Sci 93, 111-115] (kindly provided by D. Jans, John Curtin School of Medical Research, Canberra, Australia) were grown at 2×YT medium supplemented with 100 μg/ml of Ampicillin at 37° C. with shaking (225 rpm,) until reaching an OD₆₀₀ of 0.9. Protein expression was induced by 0.1 mM IPTG and the culture was further grown overnight (about 16 hrs) at 28° C., with shaking. Following centrifugation (12,000×g, 15 min, Sorvall),the pellet obtained was resuspended in 10 mM Tris-HCl buffer, to a final volume of 1/100 of the original volume of the culture, and sonicated for 6×1 min cycles. The turbid solution obtained was centrifuged as above, the supernatant was then added to pre-swelled Glutathione-Agarose resin (Sigma, No. G4510), in about 1 ml of resin/20 ml solution and incubated with rotation overnight at 4° C. The agarose resin was then intensively washed with 1×PBS (50-100 ml of 1×PBS per 1 ml resin), and the bound Vpr-GST was eluted with 1×PBS/30 mM free glutathione/3 mM DTT, by incubation with rotation at 4° C. for 1 hour. The eluted protein was concentrated by VivaScience concentrators, MWCO 30,000 (Sartorius) and stored at −20° C.

Fluorescence Labeling of Vpr-GST

The Vpr-GST fusion protein eluted from the Glutathione-Agarose resin was concentrated by VivaScience concentrators (MWCO 30,000) to a final concentration of 2.5-3.0 mg/ml, dialyzed for 2-3 hours against 1×PBS at 4° C. and labeled with rhodamine-maleimide Molecular Probes) according to manufacturer's instructions. Following 2 hours of incubation at room temperature with rotation, the labeled Vpr-GST fusion protein (FL-Vpr-GST) was purified using Sephadex G-25 fine (Pharmacia). The FL-Vpr-GST was then concentrated again using VivaScience concentrators (MWCO 30,000) and stored at −20° C.

General Methods of Molecular Biology

A number of methods of the molecular biology art are not detailed herein, as they are well known to the person of skill in the art. Such methods include PCR cloning, expression of cDNAs, analysis of recombinant proteins or peptides, transformation of bacterial and yeast cells, transfection of mammalian cells, and the like. Textbooks describing such methods are, e.g., Sambrook et al. (1989) Molecular Cloning, A Laboratory Manual, Cold Spring Harbor Laboratory, ISBN: 0879693096; F. M. Ausubel (1988) Current Protocols in Molecular Biology, ISBN: 047150338X, John Wiley & Sons, Inc. Furthermore, a number of immunological techniques are not in each instance described herein in detail, as they are well known to the person of skill in the art. See, e.g., Harlow and Lane (1988) Antibodies: a laboratory manual. Cold Spring Harbor Laboratory.

### EXAMPLES

The inventors have applied the phage display technology and used a scFv antibody library for isolation of scFv antibodies against the VprN sequence. The library used has >10⁸ of diversity and was built using the repertoire of 49 in vitro rearranged human VH genes, with random nucleotide sequence of 4-12 amino acids encoding for CDR3, linked to the VL domain by a flexible polypeptide (constructed and obtained from Dr. A. Nissim). The resulting scFv antibody fragment was displayed on the minor coat protein pIII of the M-13 bacteriophage Rasched, I., and E. Oberer (1986) Microbiol. Rev. 50(4), 401-27; Johnsson K., and L. Ge (1999) Curr Top Microliol Immunol 243, 87-105]. The inventors were able to select more than 40 phages bearing specific anti-VprN scFv antibodies. From these, 3 scFv antibodies were arbitrarily chosen. These three antibodies specifically recognized the VprN peptide but not peptides bearing a “mutated” VprN or other NLS's, such as that of the SV40 large T antigen (SV40 NLS). The selected anti-VprN antibodies strongly inhibited nuclear import mediated by the VprN peptide but not by the SV40 large T antigen NLS. Furthermore, the anti-VprN scFv antibodies were able to specifically bind and inhibit nuclear import of a full length Vpr-GST fusion protein. These results are described in greater detail in the following examples.

### Example 1

### Selection of Phage Clones Binding Specifically to the VprN Peptide

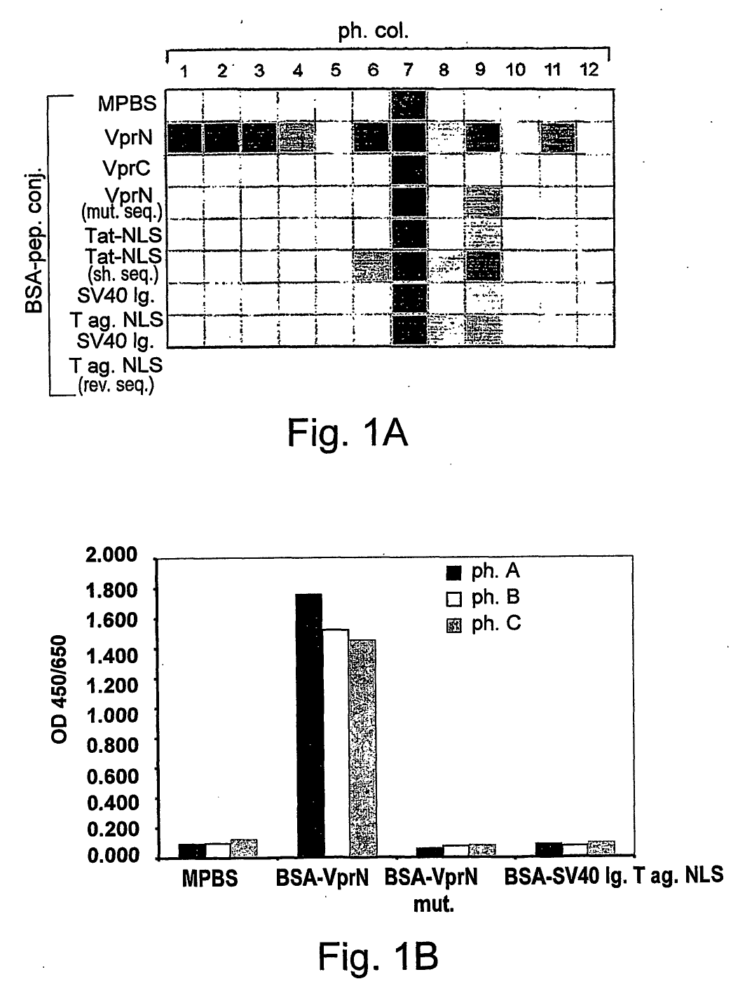

VprN-BSA conjugates have been used by the inventors as a target to select phage particles from a semi-synthetic phage display ScFv antibody library, which specifically bound the VprN moiety of the conjugate [Harrison (1996) id ibid.; Nissim (1994) id ibid.], as schematically indicated above. In order to assure the selection of VprN-binders and exclude the BSA-binders, phages selected by the first panning round were incubated again with surface bound BSA molecules and the unbound particles collected. Using this strategy the percentage of specific VprN binders was around 7% of the total particles screened, as compared to only about 0.5% obtained when the BSA panning step was omitted (Step II in the schematic). This two-round selection procedure assured the isolation of phage particles which possessed selective and specific affinity for the VprN sequence. This was confirmed by experiments using an ELISA assay system (Step III and Step IV in the schematic and FIG. 1A). Phage particles which exhibited high and selective binding to VprN-BSA, and not to the other NLS-BSA conjugates, were considered specific binders. BSA conjugates carrying peptides with the following sequences were used to detect unspecific binders: the NLS of the SV-40 large T-antigen (SV40 NLS) or its sequence in a reversed order (“revertant”), the ARM sequence which serves as the NLS of the HIV-1 Tat protein or its short fragment (Tat “short”), a sequence derived from the C-terminus of the Vpr protein (VprC) or a VprN “mutant” sequence (FIG. 1).

The results depicted in FIG. 1A show that indeed several colonies (1-4, 11) showed specific binding to the VprN-BSA conjugates (or rather to the VprN part of it), while others (colonies 6,8,9) either were able also to bind other NLS-BSA conjugates, or (colonies 5,10,12) did not bind any of the target molecules used. In these experiments, over 40 specific binders, namely phages which bind only to VprN and not to other NLS's have been selected (data not shown). Quantitative estimation of the binding abilities of three arbitrary chosen phage colonies (designated Phages A, B, C), characterized by strong and specific binding to the target sequence, are shown in FIG. 1B. As can be seen, all the three colonies have about ten fold higher binding abilities to the VprN then to its “mutant” (which differs from the “wild type” peptide only by three amino acids) or to an unrelated NLS, as the NLS from the SV40 large T-antigen.

It is possible that the binding of the selected phages to VprN may be promoted, fully or partially, by the random insert in the CDR3 loop within the ScFv domain. Therefore, it was of interest to elucidate the amino acid sequence of this insert and study the binding abilities of synthetic peptides carrying this sequence. Following PCR of the scFv domain within the 40 VprN-BSA binding phages, the resulting nucleotide fragments were sequenced, and the corresponding amino acid sequence determined. Using ClastalW [Thompson, J. D. et al (1994) Nucl. Acids Res. 22, 4673-4680] and MEME [Bailey, T. L., and C. Elkan (1994) Fitting a mixture model by expectation maximization to discover motifs. Proceedings of the Second International Conference on Intelligent Systems for Molecular Biology 28-36] algorithms, no common determinants (consensus sequence) could be elucidated among the various sequences determined.

Out of the 40 CDR3 random insert sequences elucidated, the amino acid sequences and the VH types of the three previously mentioned phages (A,B,C) are depicted in Table 1.

TABLE 1

| | Corresponding | Peptide | Name of the |
|---|---|---|---|
| Sequence | phage | designation | sequence |
| ISSD | A | a | SEQ. ID. NO. 4 |
| AFMKSGKRFIH | B | b | SEQ. ID. NO. 5 |
| HFHYKGKLQTF | C | c | SEQ. ID. NO. 6 |

### Example 2

### A Synthetic Peptide Derived from the CDR3 Insert of an Anti-VprN scFv Antibody Binds the VprN-BSA Conjugate but Fails to Inhibit its Nuclear Import

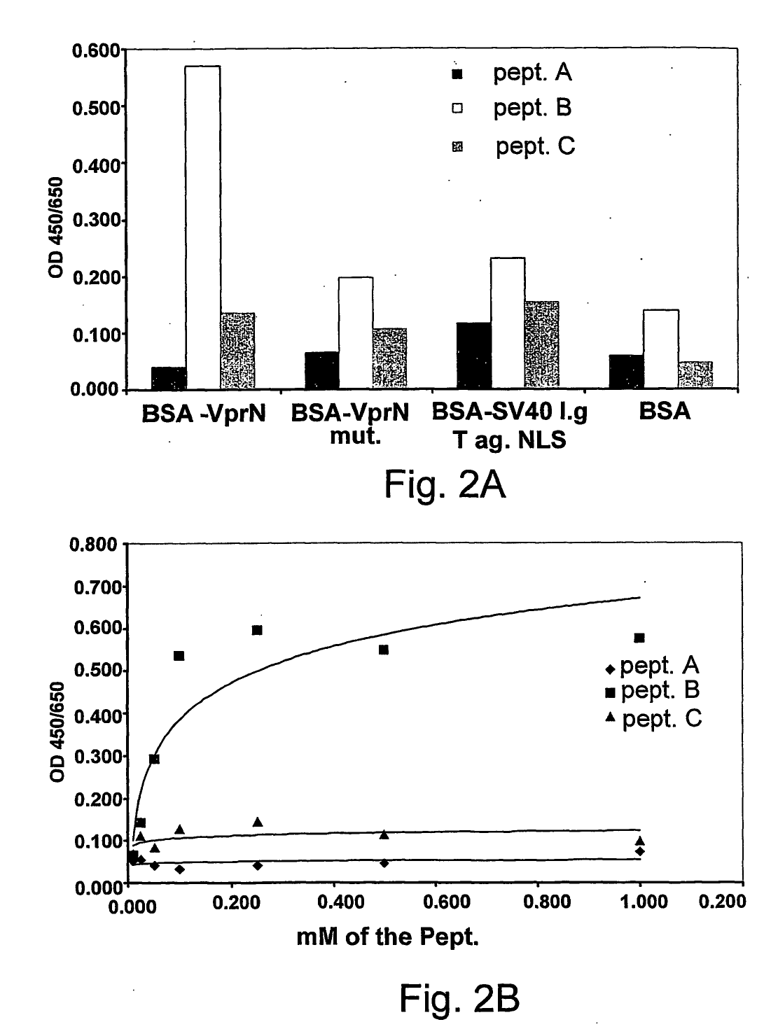

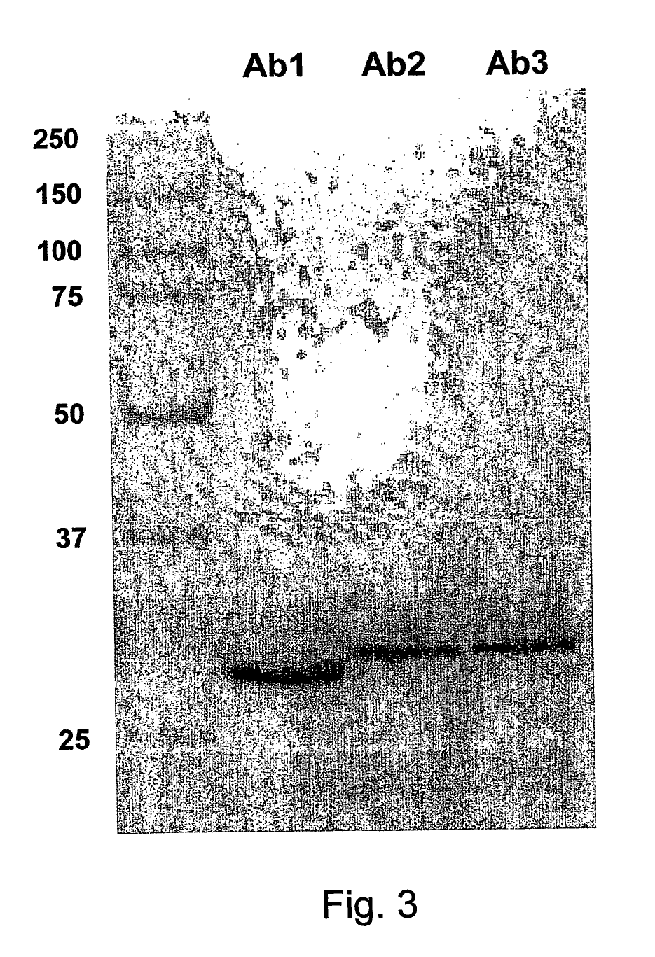

In order to find out whether the binding observed with the phage particles A, B and C to VprN is promoted by the CDR3 random inserts, peptides corresponding to these domains have been synthesized and designated as peptides “a”, “b” and “c” respectively (Table 1). Out of the three synthetic peptides only peptide “b” exhibited specific binding to the VprN-BSA conjugate as was determined by an ELISA assay system (FIGS. 2A and 3B). Peptides “a” and “c”, whose sequences are based on the nucleotide insert of the two other phage binders (A and C) failed to recognize the VprN-BSA conjugate (FIGS. 2A and 2B). The binding capabilities of these two peptides to VprN did not exceed their binding to the VprN “mutant” or SV40 NLS, all of which were very close to values obtained by the background control (FIG. 2A). Binding of peptide b to the VprN sequence was able to reach saturation, and the maximum values were at a peptide concentration of 1 mM (FIG. 2B). However binding of peptide “b” to VprN was only about 2-3 fold higher than that observed with VprN “mutant” or SV40 NLS, implying that the interaction between peptide b and VprN is of low avidity (FIG. 2A).

Peptide “b”, even at relatively high concentrations (peptide/transport substrate 20-40/1 mole/mole), did not cause any significant reduction in the import of Fl-BSA-VprN or Fl-BSA-SV40 NLS conjugates into nuclei of permeabilized HeLa cells (not shown). At higher peptide/transport substrate ratios this peptide caused nonspecific effects.

### Example 3

### Anti-VprN ScFv Antibodies Possess High and Specific Binding Abilities to the VprN Peptide

Following the failure of the synthetic peptides “a” and “c” to bind to the VprN or of peptide “b” to inhibit nuclear import, attempts have been made to obtain ScFv anti-VprN antibodies from phages A, B and C, in order to determine whether such ScFv antibodies possess anti-NLS activity.

The three phage clones (A, B and C), were propagated and the respective ScFv antibodies expressed in the non-suppressor HB21521 bacterial strain. The antibodies were then purified through Sepharose columns with covalently attached VprN-BSA conjugates (see Experimental Procedures). After affinity chromatography, highly pure ScFv antibodies with the appropriate M.W. of 30 kDa were obtained from phages A, B and C (FIG. 3) and designated as Ab1, Ab2 and Ab3, respectively.

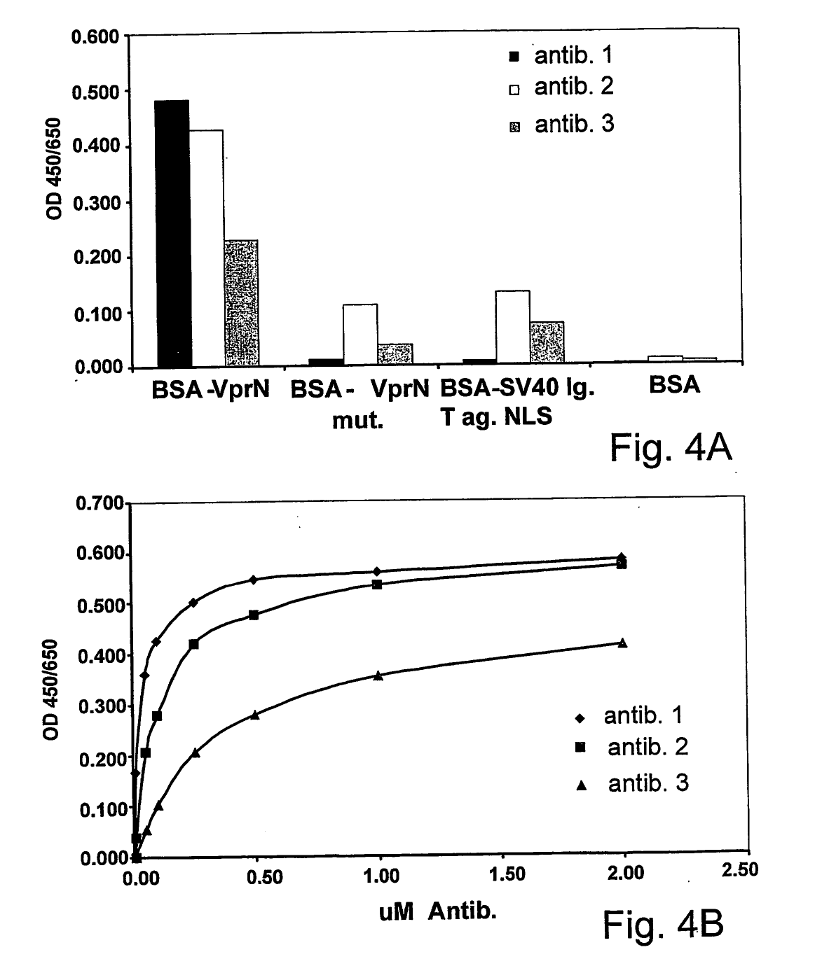

Binding studies revealed that all three purified antibodies were able to specifically bind to the VprN-BSA conjugate (FIG. 4A). Binding reached saturation, with maximum binding values at a concentration of 1.0 μM for Ab1 and Ab2 and at 2.0 μM with Ab3 (FIG. 4B). All the three antibodies failed to show any significant binding to non-conjugated BSA molecules indicating specific attachment to the VprN moiety of the VprN-BSA conjugate. The highest binding avidity and specificity was exerted by Ab1 which showed about 100 fold higher binding values to the VprN-BSA conjugate than to conjugates bearing the VprN “mutant” or the SV40 NLS (FIG. 4A). Ab2 and Ab3 displayed lower binding specificities, which was consistent with they displaying high binding values also to VprN, and although low, some binding to the VprN “mutant” and to the SV-40 NLS (FIG. 4A).

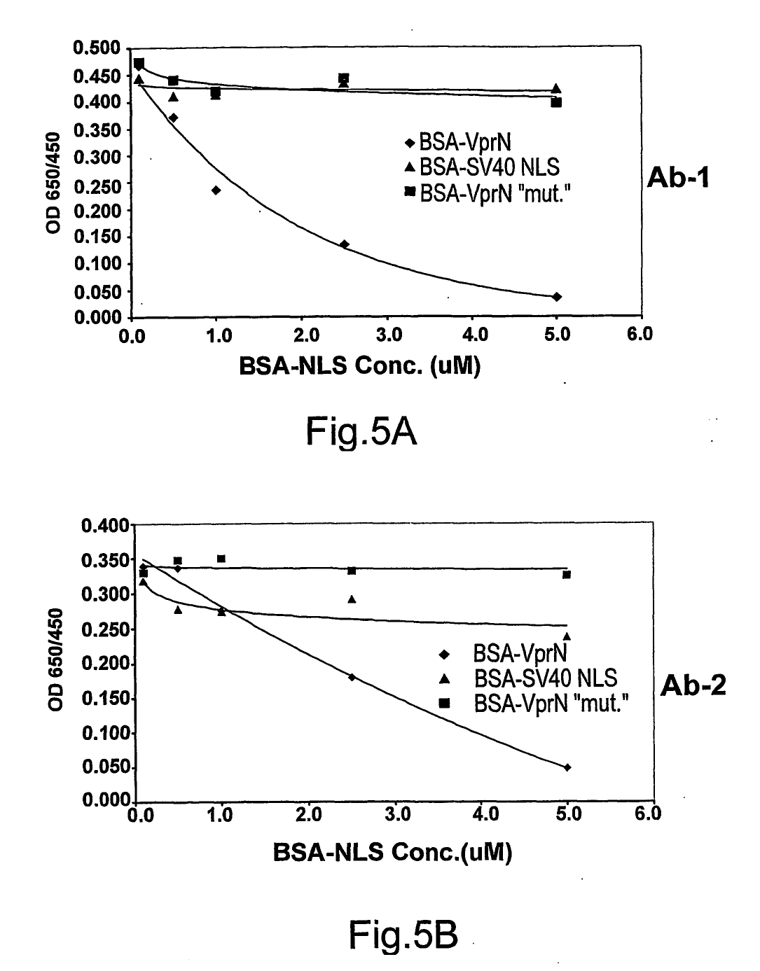

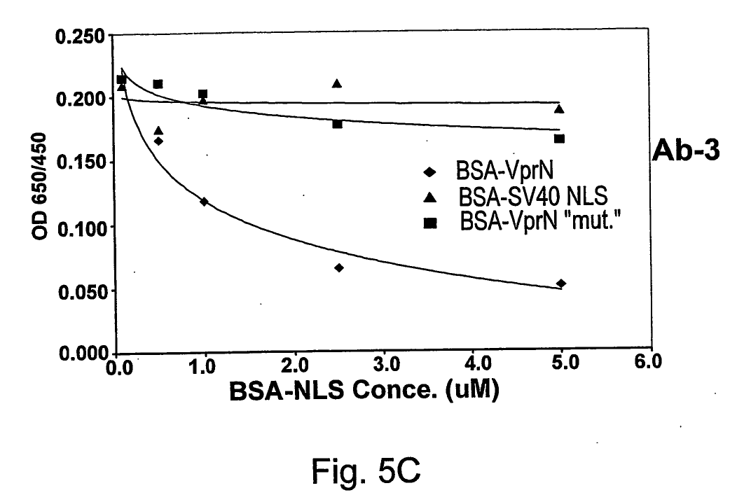

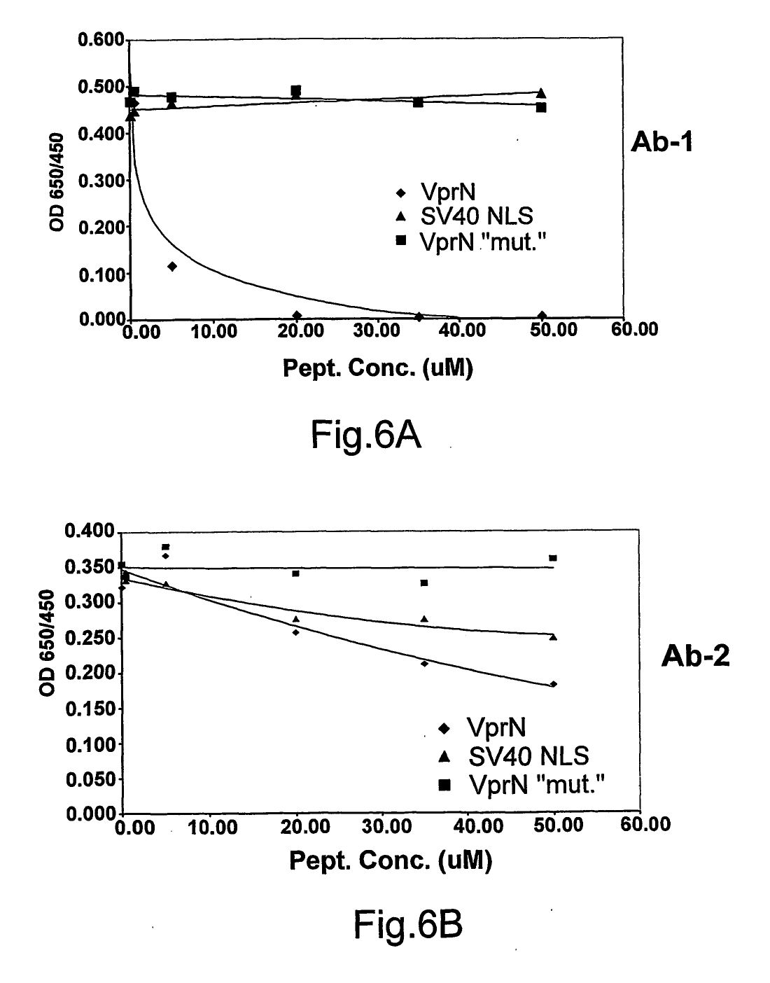

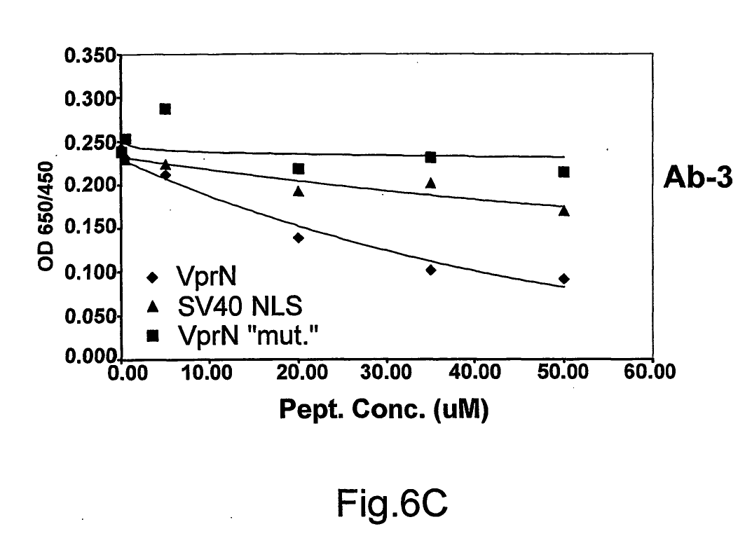

Binding of the three antibodies to its antigen, the VprN-BSA molecule, was inhibited by the addition of soluble ligands, namely by VprN-BSA conjugates or by free VprN peptides (FIGS. 5 and 6). The results in FIG. 5 show that the VprN-BSA conjugates compete with and block the specific binding of the antibodies much better than the free VprN peptides (FIG. 4). As can be seen, binding of Ab1 to its antigen was inhibited by 90% in the presence of about 5 μM of VprN-BSA, while more than 15 μM of free VprN peptide was needed to exert the same inhibition. The high inhibitory effect of the VprN-BSA conjugate, as compared to that of the free VprN peptide, can be attributed to the fact that each BSA molecule bears about 3-4 covalently attached VprN peptides [Friedler, A. et al. (1999) J Mol Biol 289, 431-437]. No or low inhibition was observed either by the addition of the VprN “mutant”, by its BSA-conjugate, by the SV40 NLS peptide, or by its conjugate (FIGS. 4 and 5).

### Example 4

### Inhibition of VprN-mediated Nuclear Import by anti-VprN ScFv Antibodies

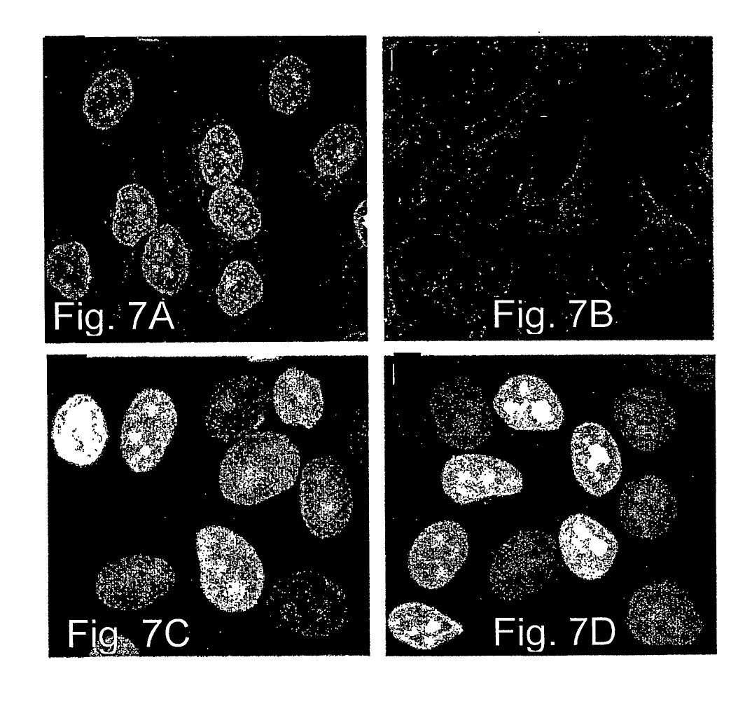

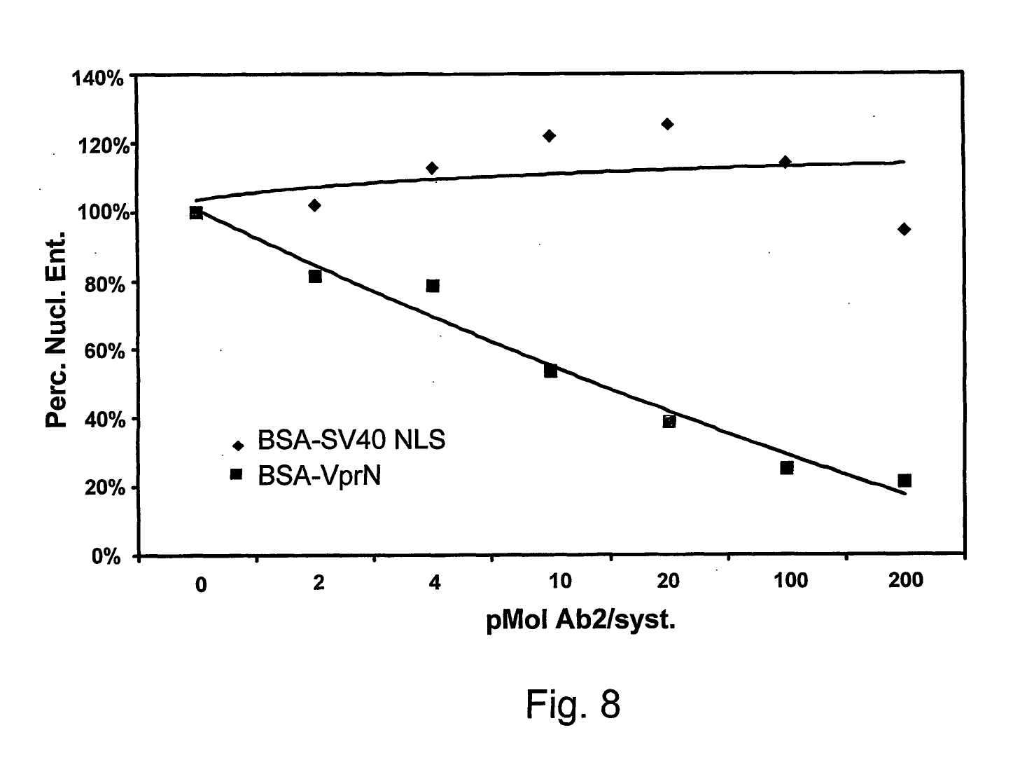

Active nuclear import mediated by the VprN sequence was strongly and specifically inhibited by all the three anti-VprN antibodies. Inhibition was observed when nuclear import was studied either with permeabilized HeLa (FIG. 6) or Colo-205 (FIG. 7) cells, as well as with intact microinjected COS cells (FIG. 8).

Ab2 inhibition of nuclear import of FL-BSA-VprN conjugates in permeabilized HeLa cells was monitored by confocal fluorescence microscopy (FIG. 6). Identical inhibitory effects were obtained with Ab1 or Ab3. The specificity of Ab2 was confirmed in the experiment where no inhibition was observed when the FL-BSA-SV40 NLS conjugate was used as a transport substrate (FIGS. 6C and 6D). The results summarized in Table 2 confirm the inventors' previous observations that nuclear import of VprN-BSA conjugates, in contrast to SV40 NLS-BSA conjugate, in permeabilized cells did not require the addition of soluble cytosolic factors [Karni et al. (1998) id ibid.]. On the contrary, addition of a reticulocyte extract, used routinely as a source of soluble factors needed for nuclear import, caused inhibition of VprN-BSA conjugates translocation into the nuclei of HeLa cells (Table 2). Nuclear import of the BSA-VprN conjugate was characterized by the same features that characterize specific and active nuclear import [Goldfarb, D. S. et al. (1986) Nature 322, 641-644; Karni et al. (1998) id ibid.]. In other words, it was temperature dependent, partially inhibited by GTP-γ-S and ATP depletion, as well as by wheat germ agglutinin (WGA) (Table 2).

Quantitative estimation of nuclear import using an ELISA-based assay system revealed that the inhibition exerted by Ab2 exceeded 80% for BSA-VprN, and was close to 0% for BSA-SV40-T-Ag NLS conjugates (FIG. 7).

To study the inhibitory activity of the antibodies within the environment of an intact cell, Ab2 was co-microinjected with FL-BSA-VprN or FL-BSA-SV40 NLS conjugates into the cytoplasm of cultured COS cells. As can be seen in FIG. 8, microinjected FL-BSA-VprN, as well as FL-BSA-SV40 NLS conjugates readily accumulated within the nuclei of the microinjected cells (FIGS. 8A and 8C). However, when these transport substrates were co-microinjected with Ab2, complete inhibition of nuclear accumulation was observed only in cells microinjected with the FL-BSA-VprN, but not with the FL-BSA-SV40 NLS conjugates (FIGS. 8B and 8D).

### Example 5

Anti-VprN ScFv Antibodies Block Nuclear Import of the Full Length Recombinant Vpr-GST Fusion Protein

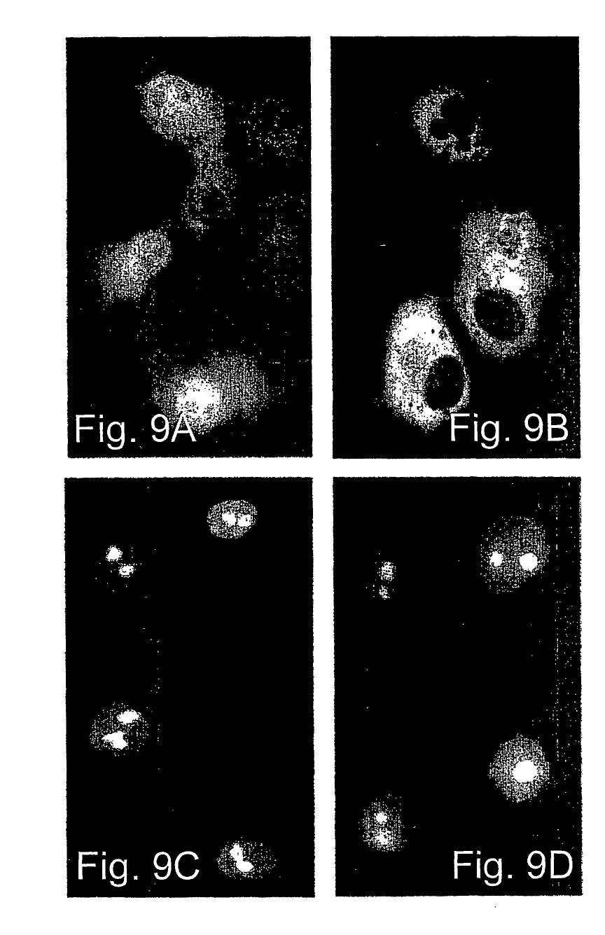

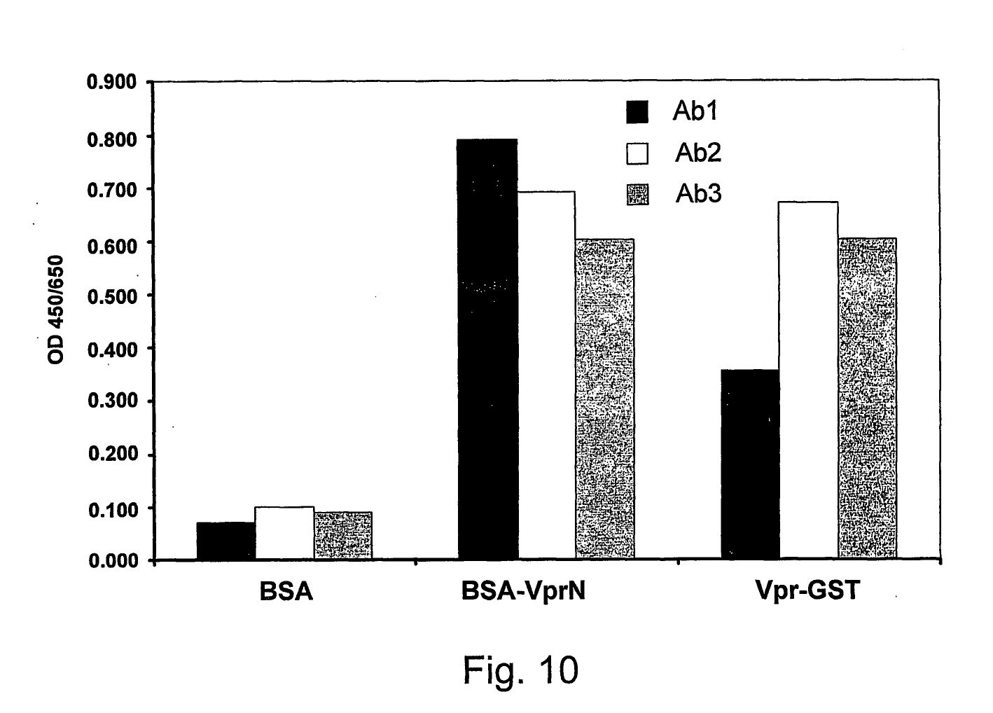

The results in FIG. 9 demonstrate that when using the VprN peptide as a target, the scFv antibodies recognized also the full length recombinant Vpr-GST fusion protein. As can be seen, the binding affinities of Ab2 and Ab3 to the Vpr-GST fusion protein were very close to those observed with the VprN-BSA conjugate, while Ab1 showed a somewhat lower binding capacity to the recombinant protein, as compared to that observed with the VprN-BSA conjugate. Specificity of the binding can be inferred from the experimental data shown in FIG. 10. The binding of all three antibodies to the Vpr-GST was strongly inhibited by the addition of VprN peptide (FIG. 10A) and of VprN-BSA conjugate (FIG. 10B), but no or little inhibition was observed when VprN “mutant” peptide or its BSA conjugate were added (FIG. 10). This strongly indicates that the domain with which the antibodies interact within the full length protein is VprN, i.e. the domain including amino acids 17-34 of the Vpr protein. FIG. 10 also shows that about ten fold lower concentrations of the VprN-BSA conjugate than that of the VprN peptide were required to block the binding of the antibodies to the Vpr-GST. This observation is consistent with the results of the competition experiments shown in FIGS. 4 and 5.

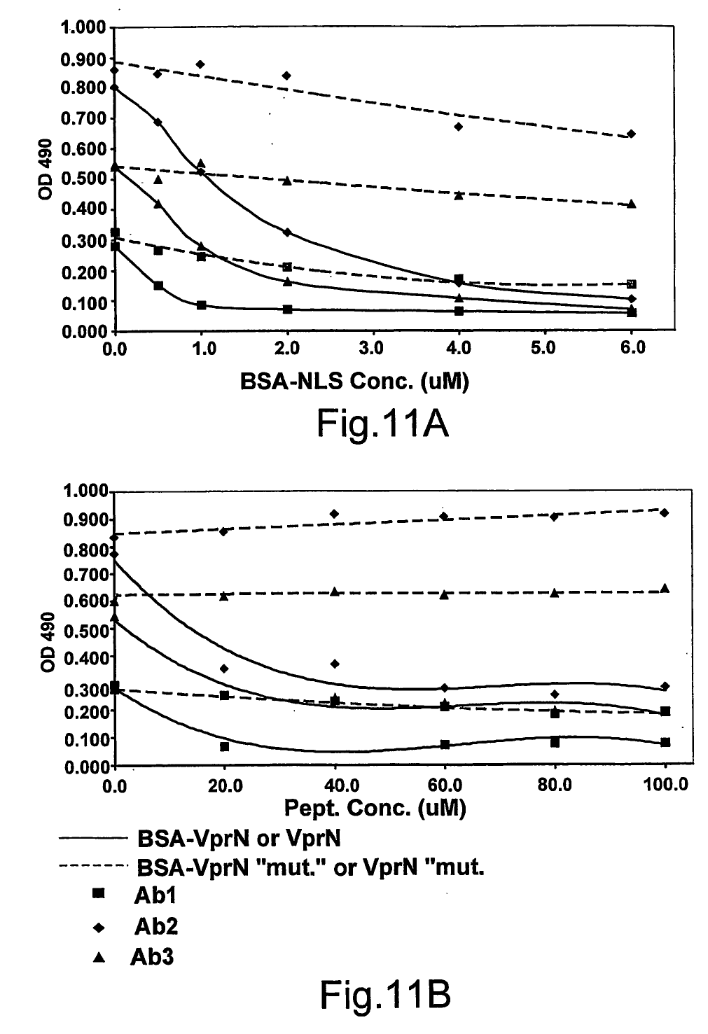

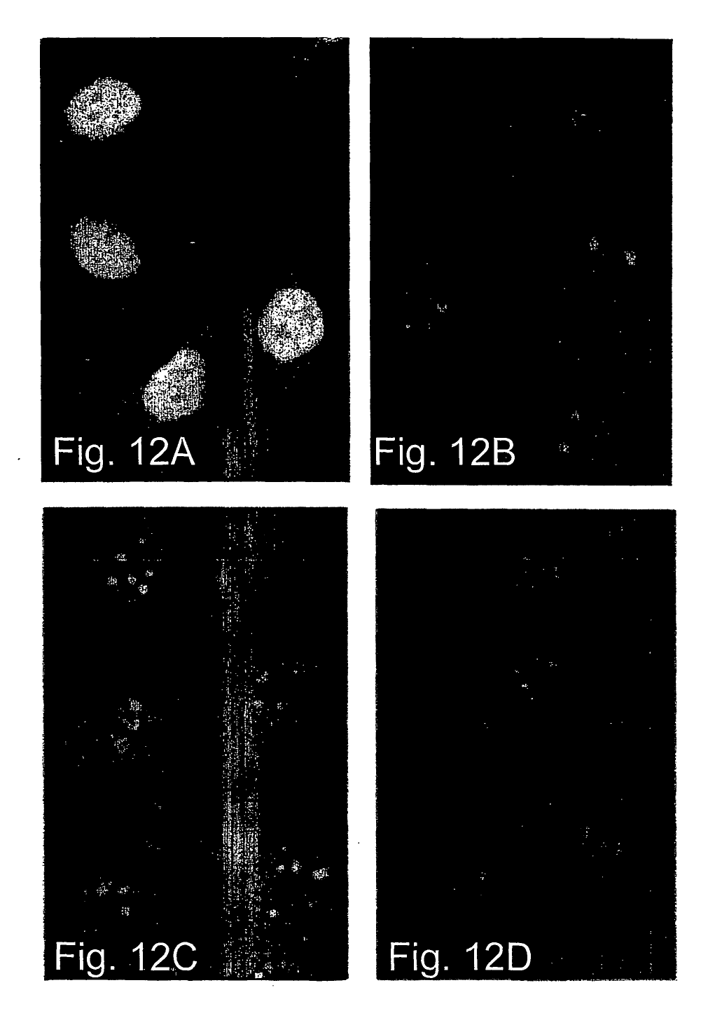

The inventors have also shown that similar to the inhibition of nuclear import observed with VprN-BSA conjugates, import of the fluorescently labeled recombinant Vpr-GST fusion protein was inhibited by Ab2 (FIG. 11A and 11B). The results shown in FIG. 11 lead to the conclusion that nuclear import of the recombinant Vpr-GST protein is receptor-mediated, and occurs via the nuclear pore complex, since it was inhibited by 5-fold excess of the unlabeled Vpr-GST fusion protein and by WGA (FIGS. 11C and 11D). Nuclear import of Vpr-GST was reliant on features characteristic to active nuclear import, namely it was ATP-dependent, partially inhibited by GTP-γ-S and its extent was reduced following incubation at 4° C. (data not shown).

### Example 6

### The fd Bacteriophage Recognizes the ARM Sequence of the HIV-1 Tat Protein

During the attempt to select, from a phage display peptide library, peptides which specifically recognize the NLS (ARM) sequence of the HIV-1 Tat protein, the inventors have observed that the bacteriophage particles themselves strongly bind to this NLS sequence.

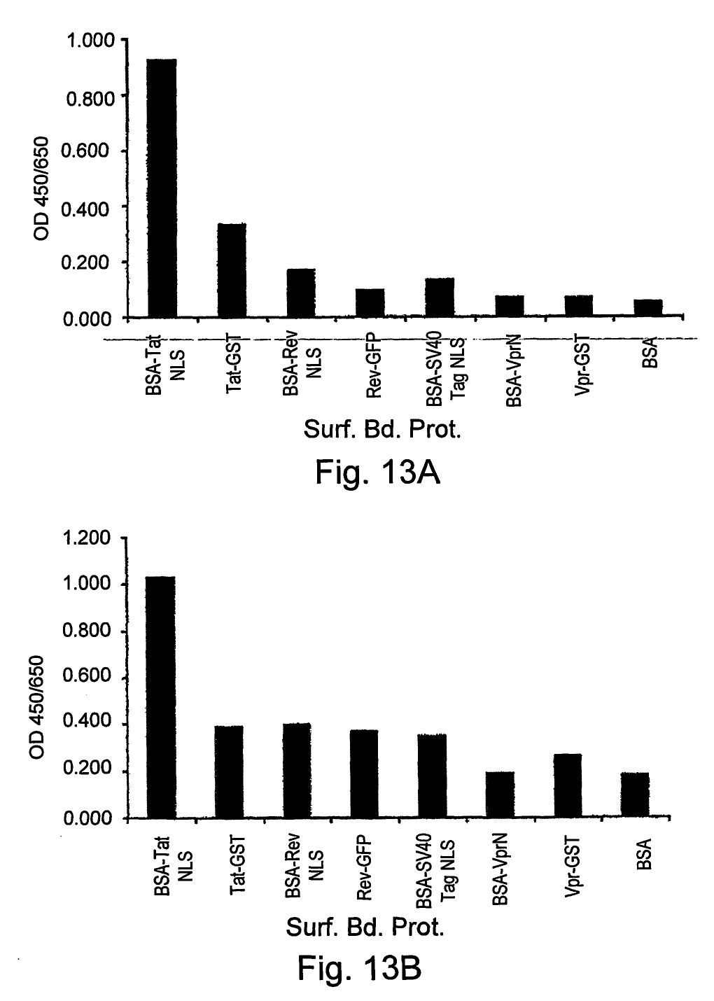

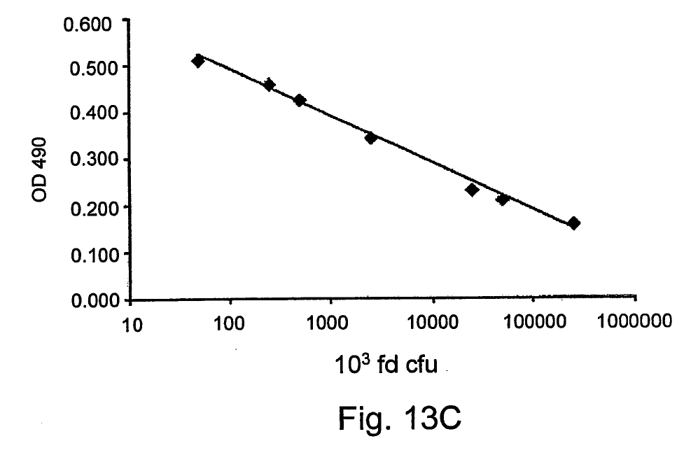

Using an ELISA based assay system, a high degree of binding was observed following the addition of the fd phage particles to plates coated with Tat-NLS-BSA conjugates (FIG. 13A). Very little, if any, binding was obtained when the bacteriophage was incubated with plates coated with BSA molecules alone, clearly indicating that the binding observed was mediated by the Tat-NLS (ARM) (FIG. 13A). In most of the experiments, Tat-NLS-BSA conjugates were used as a ligand since these conjugates were the substrate for the nuclear import assay (see FIG. 14). A relatively high degree of binding was observed to the Tat-GST fusion protein. Specificity of bacteriophage-Tat NLS (ARM) interaction can also be inferred from the results showing that very little, if any, binding was obtained to conjugates bearing the NLSs of the HIV-1 Vpr and Rev proteins as well as that of the SV40 T-antigen (FIG. 13A). Also a low degree of binding was obtained to plates coated with Vpr-GST or the Rev-GFP fusion proteins.

### Example 7

### The N-terminus of the Phage P8 Protein Promotes the Interaction between the fd Bacteriophage and the Tat-ARM

The fd bacteriophage is composed of 5 structural proteins, out of which the coat protein p8 is the major one and is present at approximately 3,000 copies per phage. The other four structural proteins exist at only about 5 copies per particle. Therefore, it is likely that the specific interaction between the phage particles and the Tat ARM described above is promoted by the exposed part of the p8 protein namely by its N-terminal domain [F. C. L. Almeida and S. J. Opella (1997) J. Mol. Biol. 270, 481-495]. A synthetic peptide bearing the first twenty amino acids of the p8 protein was thus synthesized (sequence below), designated as NTP8 peptide (SEQ. ID. NO.16) and its interaction with the Tat-ARM studied.

SEQ. ID. NO.16:
AEGDDPAKAAFDSLQASATE

As is evident from the results shown in FIG. 13B, the NTP8 peptide itself was able to interact with immobilized Tat-BSA conjugates showing a binding degree that was about 2-3 fold higher than that observed with any other immobilized molecule, including BSA. Interaction with other BSA-NLS conjugates and other recombinant proteins was at background level (FIG. 13B).

The binding of the NTP8 peptide to the immobilized Tat-BSA conjugates was competitively inhibited by the addition of increasing concentrations of the phage particles (FIG. 13C).

### Example 8

### Inhibition of the Tat-ARM Biological Functions by the fd Bacteriophage

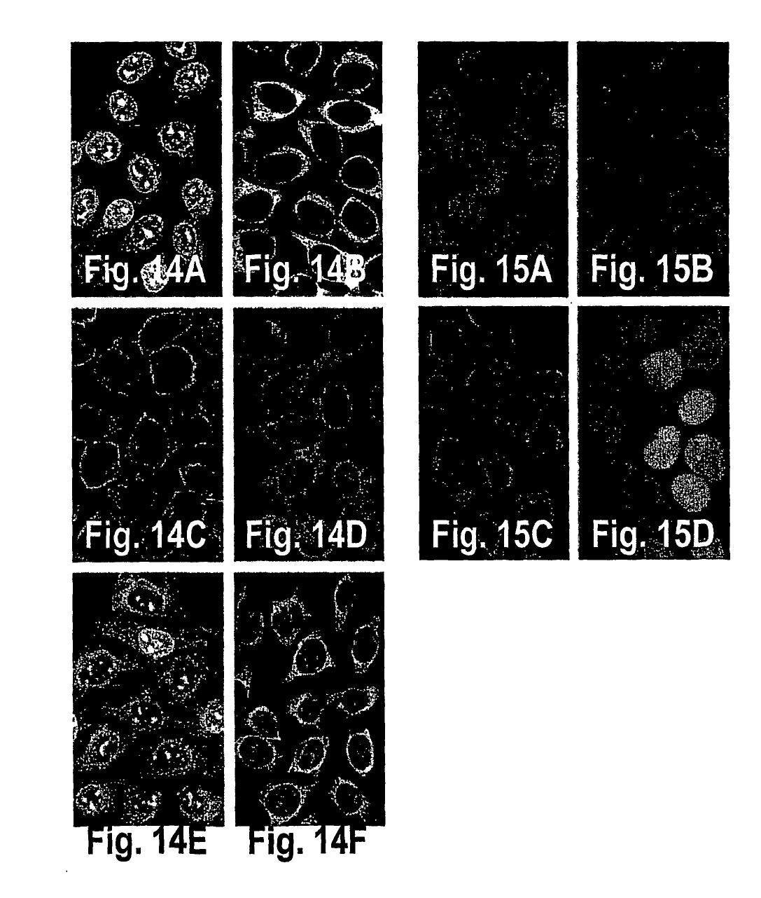

The results in FIG. 14D show that the fd bacteriophage strongly blocked Tat-ARM mediated nuclear import. As can be seen nuclear import of the FL-BSA Tat ARM conjugates was characterized by the same features that characterize active nuclear import, namely it was temperature dependent (FIG. 14B) and inhibited by WGA (FIG. 14C), as well as by the addition of excess free Tat-ARM peptide (FIG. 14F), but not by the SV40 Tag NLS peptide (FIG. 14E). The view that the inhibition of the nuclear import observed is due to the specific interaction of the fd bacteriophage and the Tat-ARM is further supported by the results showing that the VprN mediated nuclear import was not inhibited by the addition of the phage particles (FIG. 15D).

1-26. (canceled)

27. A substance that specifically binds at least one of the N-terminal domain of Vpr (amino acids 17-34) or its functional fragment or its derivative, the nuclear localization signal (NLS) of Vpr or its derivative, the HIV-1 protein Tat or its NLS, or the ARM sequence of Tat.

28. The substance of claim 27 that specifically binds the N-terminal domain of Vpr (amino acids 17-34) or its functional fragment or its derivative, the nuclear localization signal (NLS) or Vpr or its functional fragment or its derivative, or the ARM sequence of Tat.

29. The substance of claim 27 that specifically binds the HIV-1 protein Tat or its NLS or the ARM sequence of Tat.

30. The substance of claim 27 that specifically binds the N-terminal domain of Vpr (amino acids 17-34) or the nuclear localization signal (NLS) of Vpr, or functional fragments or derivatives of said molecule.

31. The substance of claim 27 that specifically binds the HIV-1 protein Tat or its NLS.

32. The substance of claim 27 that specifically binds the ARM sequence of Tat.

33. The substance according to claim 27, wherein said substance, or fragments thereof, is selected from any one of a naturally occurring, synthetic or recombinant antibody, scFv, Fv, Fab′, Fab, diabody, linear antibody, F(ab′)₂ antigen binding fragment of an antibody, a protein, a peptide and a small molecule.

34. The substance according to claim 33, wherein said substance is a scFv.

35. The substance according to claim 34, wherein said scFv is a recombinant scFv.

36. The substance according to claim 28, wherein said substance has a CDR3 region having an amino acid sequence of any one of SEQ. ID. NO. 1, SEQ. ID. NO.3, and SEQ. ID. NO.5.

37. The substance according to claim 36, wherein said amino acid sequence is encoded by the nucleic acid sequence of SEQ. ID. NO. 2, SEQ. ID. NO.4, and SEQ. ID. NO. 6.

38. The substance according to claim 29, wherein said substance is the p8 protein of the fd bacteriophage.

39. The substance according to claim 38, wherein said substance is a bacteriophage fd p8-derived peptide.

40. The substance according to claim 39, wherein said peptide has the amino acid sequence as denoted by SEQ. ID. NO.16.

41. A composition comprising at least one substance, wherein said substance is as defined in claim 27, and optionally further comprising pharmaceutically acceptable diluents, additives and carriers.

42. A method of specifically inhibiting the import of a NLS-containing molecule into a nucleus of a cell, by contacting said cell with at least one substance as defined in claim 27.

43. A method of inhibiting the import of Vpr into the nucleus of a cell, by contacting said cell with at least one substance as defined in claim 30.

44. A method of inhibiting the import of Tat into a nucleus of a cell, by contacting said cell with at least one substance as defined in claim 29.

45. A method of inhibiting the import of the pre-integration complex (PIC) into a nucleus of a cell, by contacting said cell with at least one substance as defined in claim 30.

46. A method of inhibiting viral infection by administering at least one substance as defined in claim 27 to an organism in need.

47. The method according to claim 46, wherein said organism is any one of a plant and a mammal.

48. A method of inhibiting cell proliferation, oncogenesis and autoimmune response, by administering at least one substance as defined in claim 27 to an organism in need.

49. The method according to claim 48, wherein said organism is a mammal.

50. A method of conferring immunity against a viral infection, by administering to a subject in need a substance as defined in claim 27.
